(19) United States
(12) Patent Application Publication
(10) Pub. No.: US 2008/0072899 A1
(43) Pub. Date: Mar. 27, 2008

(54) SYSTEM AND METHOD FOR DELIVERING MEDICATIONS

(75) Inventors: William F. Niland, Arnold, MD (US); Robert Storey, Arnold, MD (US); Owen S. Bamford, Linthicum, MD (US); Felino V. Cortez, Bowie, MD (US); David Lain, Easton, MD (US)

Correspondence Address: RATNERPRESTIA, P O BOX 980, VALLEY FORGE, PA, 19482-0980, US

(73) Assignee: Vapotherm, Inc., Stevensville, MD (US)

(21) Appl. No.: 11/851,713

(22) Filed: Sep. 7, 2007

Related U.S. Application Data
(60) Provisional application No. 60/843,093, filed on Sep. 8, 2006.

(51) Int. Cl. A61M 16/16

(52) U.S. Cl. 128/203.16

(57) ABSTRACT

Systems and methods for delivering medication to patients are disclosed. The system includes an infuser including a membrane. The infuser entrains a fluid from a first side of a membrane and a medication into a gas on a second side of the membrane to form a medicated humidified gas. A delivery system is coupled to the infuser. The delivery system is configured to deliver the medicated humidified gas to the patient. The method includes entraining a fluid on a first side of a membrane and a medication into a gas on a second side of the membrane to form a medicated humidified gas and delivering the medicated humidified gas to the patient using a delivery system.

### CROSS REFERENCE TO RELATED APPLICATIONS

The present application claims priority from U.S. Provisional Patent Application Ser. No. 60/843,093, filed on Sep. 8, 2006 entitled SYSTEM AND METHOD FOR DELIVERING MEDICATIONS, the contents of which are incorporated fully herein by reference.

### FIELD OF THE INVENTION

The present invention relates to systems and methods for delivering medication via a breathing gas.

### BACKGROUND OF THE INVENTION

The delivery of a breathing gas such as oxygen and oxygen-enriched air to the respiratory tract of a patient often results in discomfort to the patient, especially when the breathing gas is delivered over an extended period of time. In addition, airflow may become obstructed or limited due to mucosal edema, inflammation, bronchoconstriction and other primary pathophysiologic abnormalities in acute exacerbations of Chronic Obstructive Pulmonary Disease (COPD), and status asthmaticus, and other forms of respiratory distress and respiratory failure. Several inhaled systems are used today to deliver medications via the lung, including: metered-dose inhalers, dry power inhalers, aerosol nebulizers, short and long term (24 hour) aerosol nebulizers, and inhaled gas systems for inhaled nitric oxide and heliox.

### SUMMARY OF THE INVENTION

Briefly, the present invention provides a system for delivering medication to a patient. The system includes an infuser including a membrane. The infuser entrains a fluid from a first side of a membrane and a medication into a gas on a second side of the membrane to form a medicated humidified gas. A delivery system is coupled to the infuser. The delivery system is configured to deliver the medicated humidified gas to the patient.

The present invention also provides a system for delivering medication to a patient. The system comprises an infuser having a membrane. The infuser includes a medication that produces a medicated humidified gas for delivery. A delivery system is coupled to the infuser. The delivery system is configured to deliver the medicated humidified gas to the patient.

Also, the present invention provides a system for delivering medication to a patient. The system comprises means for producing a medicated humidified gas for delivery to the patient and means for delivering the medicated humidified gas to the patient.

Further, the present invention provides a method of delivering medication to a patient. The method includes entraining a fluid from a first side of a membrane and a medication into a gas on a second side of the membrane to form a medicated humidified gas and delivering the medicated humidified gas to the patient using a delivery system.

Additionally, the present invention provides a method of delivering medication to a patient. The method comprises infusing a fluid and a medication into a gas using a humidification cartridge to form a medicated humidified gas; and delivering the medicated humidified gas to the patient.

### BRIEF DESCRIPTION OF THE DRAWINGS

FIG. 1 is a block diagram displaying the main components of a system according to aspects of this invention;

FIG. 2 is a schematic representation of an exemplary embodiment of a system according to aspects of this invention; and

FIG. 3 is a schematic diagram of another exemplary embodiment of a system according to aspects of this invention.

### DETAILED DESCRIPTION OF THE INVENTION

Aspects of this invention will now be described with reference to specific examples and embodiments selected for illustration in the figures. It will be appreciated that the spirit and scope of this invention is not limited to the selected examples and embodiments, and that the scope of this invention is defined separately in the appended claims. It will also be appreciated that the figures are not drawn to any particular proportion or scale, and that many variations can be made to the illustrated embodiments without departing from the spirit of this invention.

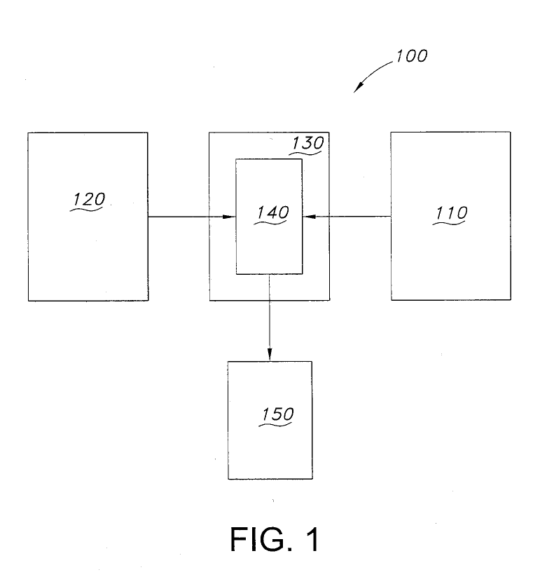

FIG. 1 depicts a block diagram of a delivery system 100 that may be used with the present invention. System 100 includes a breathing gas supply 110 and a humidification liquid supply 120. Breathing gas supply 110 and humidification liquid supply 120 are combined in a heating and humidification base unit 130. In an exemplary embodiment, unit 130 contains an infuser with a membrane, such as a vapor transfer cartridge 140 including a plurality of porous hollow fibers, having a liquid side and a breathing gas side (which are described in further detail below). Liquid from humidification liquid supply 120 is introduced from humidification liquid supply 120 into breathing gas by passing across a porous membrane in vapor transfer cartridge 140 to humidify the breathing gas. Medication may be combined with the liquid prior to its passage across the membrane and/or added to one or both sides of the membrane to be picked up by the fluid and introduced to the breathing gas. After the medication and humidity are added to the breathing gas, the humidified and medicated breathing gas is delivered to the patient, e.g., through a delivery system 150. Delivery system 150 is coupled to the infuser and may include a delivery tube and a nasal cannula.

In an exemplary embodiment, system 100 delivers the breathing gas in accordance with a high flow therapy (HFT) treatment. HFT may be defined as the delivery of breathing gas at a rate greater than 6 liters per minute (lpm) for adult patients and greater than 1 lpm for neonatal patients. Alternatively, HFT may be considered to be above 10 lpm for adults. In alternative exemplary embodiments, system 100 delivers medicated breathing gases at traditional flow rates (e.g., low flow rates), for example, to deliver 5 lpm of humidified medicated breathing gases to an adult. The amount and/or concentration of medication to be administered to the patient may be adjusted based on the gas flow rate.

In an exemplary embodiment, system 100 includes a liquid source (e.g., water) that contains a medication, or a premix of medication, held in humidification liquid supply 120. During operation of system 100, as medicated liquid passes across the membrane from the liquid side to the breathing gas side, the breathing gas is humidified and the medication is entrained in the breathing gas. As used herein, the word “entraining” and all derivations thereof is defined as the addition of medication to a breathing gas by means of small and/or large particle aerosol, vaporization, nano-particle delivery, and/or simple propulsion as droplets along a delivery tube to a patient.

In an alternative exemplary embodiment, system 100 includes vapor transfer cartridge 140 that is impregnated with a medication prior to use. The medication may be impregnated, for example, in porous hollow fibers of vapor transfer cartridge 140 that separate the humidification liquid from the breathing gas. During operation of system 100, as humidification liquid passes from the liquid side to the breathing gas side of the porous fibers of vapor transfer cartridge 140, medication is released from vapor transfer cartridge 140 into the breathing gas for delivery to the patient through delivery system 150. Vapor transfer cartridge 140 may be removable from and replaceable into base unit 130. Such a configuration allows medication to be resupplied to system 100 by replacing a medication depleted cartridge with a medication enriched cartridge.

Without being bound to any particular theory, it is believed that forcing high pressure gas flow along the fibers of a vapor transfer cartridge while also allowing liquid to transpire across the membranes of the vapor transfer cartridge, can produce fine aerosol particles and/or nano-particles by shear forces between the gas flow and the humidification liquid percolating from the liquid side to the breathing gas side of vapor transfer cartridge 140. Any medication dissolved in the humidification liquid or present within vapor transfer cartridge 140 is entrained in the breathing gas for delivery to the patient through delivery system 150.

A medication may be physically incorporated into vapor transfer cartridge 140 in a variety of ways. For example, a liquid solution of a medication may be dipped, sprayed or brushed onto the surfaces of the cartridge that contact the humidification liquid. Alternatively, a solid form of the medication may be mixed with a finely divided or liquefied polymeric resin, which resin is then molded into the cartridge components. Other processes may be used to incorporate medication into, apply a medication to, or otherwise associate a medication with humidification cartridge 140. The medication may ultimately be disposed on the exterior of the hollow fibers over which the heating and humidifying fluid flows, on the interior of the hollow fibers through which the breathing gas flows, or both. For example, for a medication having a molecular size that is too large to pass through the pore of the hollow fibers, the medication may be disposed on the interior of the hollow fibers, such that the breathing gas and the humidification fluid that has passed through the pores picks up the medication for entrainment in the humidified breathing gas.

In each of the above embodiments, the humidification liquid, with or without medication, may be optionally pumped through a heater (not shown) in base unit 130, circulated through delivery tube 150, and vaporized through vapor transfer cartridge 140. The humidification liquid may transfer heat to the breathing gas while simultaneously entraining the medication into the breathing gas, thus heating and humidifying the medicated breathing gas. Exemplary thermodynamic values of the breathing gas as it is delivered to the patient are a temperature of between about 33-43 degrees Celsius, regulated within about ±1 degree Celsius, and at least about 95 percent relative humidity. While the exemplary embodiment uses heated water for circulation through delivery tube 150 to heat and/or insulate the breathing gas, heated air may be used instead to heat and/or insulate the breathing gas. A device using heated air to heat and/or insulate the breathing gas is disclosed in U.S. Provisional Patent Application Ser. No. 60/961,020, filed on Jul. 18, 2007, which is incorporated by reference herein in its entirety.

Additionally, aspects of the Vapotherm® 2000i system (available from Vapotherm, Inc. of Stevensville, Md.) can be incorporated into delivery system 100, and the disclosure of U.S. Patent Publication No. 2003/0209246A1, which describes embodiments of an apparatus and method for respiratory tract therapy adapted to heat and humidify air and to deliver heated and humidified air to the respiratory tract of a human patient, is incorporated herein by reference in its entirety. Further embodiments relating to an apparatus and method for delivering water vapor to a gas and related filters are described in U.S. patent application Ser. No. 10/810,768, filed Mar. 26, 2004, attorney docket HQS-107US, the disclosure of which is also incorporated herein by reference. According to exemplary embodiments of the invention, vaporized gas and drugs or drug mixtures enter the patient via delivery system 150.

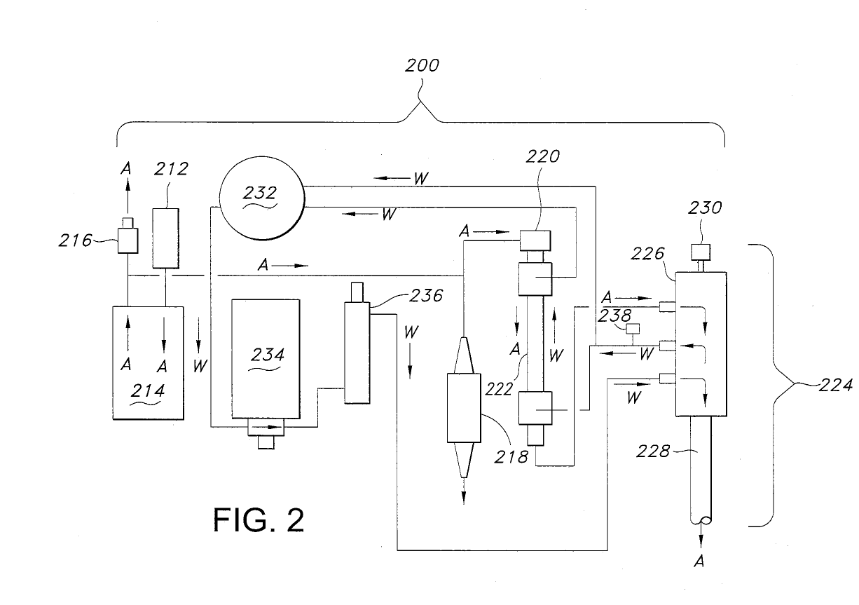

FIG. 2 illustrates a schematic representation of an exemplary delivery system 200 that may be used to deliver medication to a patient via inhaled gas. Arrows have been provided to indicate the flow of air “A” and water “W” through system 200. The flow of air, water, and medication in system 200 will now be described.

Where the breathing gas is air, air introduced into system 200 passes through a vacuum muffler 212. An air compressor 214 pressurizes the air downstream of vacuum muffler 212. A variety of air compressors can be used, and such air compressors are well known in the art. One example of a suitable air compressor is manufactured by Thomas Compressors of Norcross, Ga. and sold under the model number 007CA13F. Other compressors can be substituted. A pressure relief valve 216 is provided downstream of air compressor 214 in order to release excessive air pressure. While a compressor 214 is disclosed, those skilled in the art will recognize that a blower or other device for moving air may be used.

Air flows from air compressor 214 through a flow control valve 218, which is used to control or regulate the air flow in system 200. Air then flows to an air filter 220 that is adapted to remove contaminants from the air so that they are not delivered to the patient's respiratory tract. Air then flows through a vapor transfer cartridge 222 and through delivery tube assembly 224. More specifically, air that has been pressurized by air compressor 214 enters connector block 226 and flows outwardly toward the patient through delivery tube 228. An inlet 230 is provided for the optional introduction of oxygen into connector block 226 in order to enrich the proportion of an additional gas, such as oxygen, in the air delivered to the patient.

Referring now to the flow of water and medication through system 200 as illustrated in FIG. 1, water and medication (jointly indicated as “W”) is introduced by means of a reservoir 232 that is fed by a water supply (not shown). A water pump 234 is used to deliver a solution of the water and medication from the reservoir 232 to a fluid heater 236 which heats the solution to a predetermined temperature or temperature range, as will be described in more detail later. The heated solution then flows into delivery tube assembly 224. More specifically, heated solution enters connector block 226 and flows into delivery tube 228. The heated solution enters a lumen of delivery tube 228 to transmit heat to breathing gas flowing through an adjacent lumen, thereby thermally insulating the breathing gas. The solution then returns from delivery tube assembly 224 into the housing of supply unit. A thermistor 238 is used to monitor the temperature of the returning solution. The temperature measured by thermistor 238 is used to control water heater 236 in order to maintain the temperature of the solution within a predetermined range, such as, for example, 37 degrees Celsius±1 degree Celsius.

The returned solution flows to vapor transfer cartridge 222. This medicated solution passes across the membrane of vapor transfer cartridge 222 in order to add water vapor to the air that is flowing in counter-current arrangement through vapor transfer cartridge 222. An exemplary vapor transfer cartridge 222 is a polysulphone hollow fiber filter module having a microporous membrane that permits the passage of water vapor and medication from the heated solution into the air. More specifically, the heated solution flows through a housing of the vapor transfer cartridge 222 in contact with the outside surfaces (“liquid side”) of the hollow fiber membranes. In one embodiment, the air flows through the hollow fiber membranes (“breathing gas side”) in a direction that is counter-current to the direction of the solution in the housing of cartridge 222. Water vapor and medication pass through pores in the hollow fiber membranes from the heated solution to the air in order to humidify and medicate the air for delivery to the respiratory tract of the patient. Although a wide variety of membranes can be employed to perform this function, a hollow fiber membrane is used in the exemplary embodiment.

Suitable membranes for use with the present invention are available from Minntech, Inc. of Minneapolis, Minn. The compounds used to produce the hollow fiber membrane may be modified in order to change the chemical properties (such as polarity) of the membrane to impact diffusion of medicinal agents across the membrane from the water source into the breathing gas. For example, plastics of varying polarity or charge may be used for different therapeutic compounds to increase or decrease the rate of passage across the membrane based on interactions between the specific molecule in question and the fiber. For a medication having ionic properties, for example, the membrane may be selected from a material having a polarity such that the medication is not electrically attracted to the material of the membrane.

For an embodiment in which the medication is mixed with the humidification liquid in reservoir 232, cartridge 222 may be selected with pore openings sized to pass a particular medication. Cartridge 222 may be selectively removed from system 200 and replaced with a different cartridge depending on the drug being administered through system 200. For example, a drug or medication having a large molecular size may not be able to pass through pore openings in the hollow fibers of a cartridge sized to pass smaller molecules. Therefore, a replacement cartridge having larger pore openings may be used. Alternatively, where cartridge 222 is impregnated with medication, a medication cartridge 222 may be removed from system 200 and replaced with a medication enriched cartridge 222.

According to exemplary aspects of this invention, the “water” referred to previously in connection with FIG. 1 is optionally replaced with a medication or a medicinal solution. Vapor transfer cartridge 222 is thereby utilized to transfer or entrain the medication or solution into the flowing gas or air “A” for delivery toward the patient through delivery tube 228.

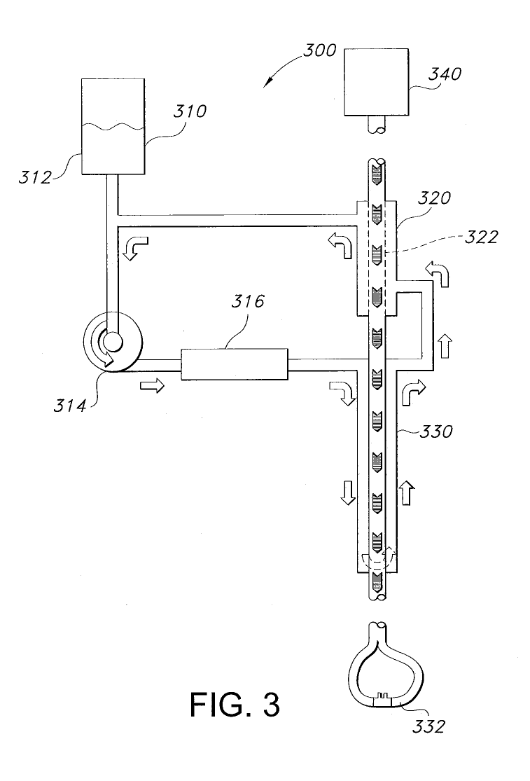

Another exemplary embodiment of a breathing system 300 for delivering medication to a patient via a breathing gas delivery system is shown schematically in FIG. 3. System 300 includes a fluid supply 310 and a breathing gas supply 340 that combine to provide heated and humidified medicated breathing gas to a patient. While system 200 discloses a compressor or blower as a source of breathing gas, system 300 uses a pressurized source of breathing gas, such as bottled gas, hospital gas, or other sources of pressurized breathing gas.

Fluid supply 310 includes a reservoir 312 that retains a fluid. The fluid may be water or the fluid may be a medicated solution. Fluid reservoir 312 may be fluidly coupled to a pump 314 that pumps the fluid from reservoir 312. Alternatively, pump 314 may be omitted and the fluid may be drained from reservoir 312 by other means, such as by gravity. Pump 314 pumps the fluid to a heater 316, where the fluid is heated to a predetermined temperature, such as, for example between about 33 to 43 degrees Celsius±1 degree Celsius. Fluid then exits heater 316, where the fluid flows through a delivery tube 330 and then through a vapor exchange medium, such as a cartridge 320. Fluid that does not diffuse through cartridge 320 recirculates through fluid supply 310 by joining fluid flow between reservoir 312 and pump 314. Optionally, although not shown, fluid may return directly to reservoir 312.

Breathing gas is provided from breathing gas supply 340. Breathing gas is directed into cartridge 320, where fluid from fluid supply 310 diffuses through cartridge 320 into the breathing gas. Cartridge 320 includes a porous hollow fiber membrane 322 that allows fluid and medication passing through cartridge 320 to be entrained into the breathing gas prior to the breathing gas flowing into delivery tube 330. Breathing gas flows through hollow fiber membrane 322 in a direction that is countercurrent to the direction of the water vapor exchange subsystem 320. Water and medication is then transferred through pores in hollow fiber membrane 322 from the heated water to the breathing gas. The passage of heated fluid and breathing gas through cartridge 320 heats as well as humidifies the breathing gas.

The medicated, heated and humidified breathing gas leaves cartridge 320 and enters delivery tube 330, where the fluid flowing through delivery tube 330 thermally insulates the breathing gas. While the embodiment shown in FIG. 3 uses liquid as the insulating fluid, other fluids, such as air, or some other fluid, may be used instead. The heated breathing gas then flows to a nasal cannula 332, from which the breathing gas is inhaled by the patient. Nasal cannula 332 may be releasably coupled to delivery tube 330.

Medications may be administered to the patient through breathing system 300 by adding medication to fluid reservoir 312 or by utilizing a reservoir filled with a medication or solution of medications. Fluid reservoir 312 may also be water or other fluid. Medication may be provided in a solid form, a liquid form, or a gaseous form that dissolves or otherwise mixes in the fluid and that can be circulated through fluid supply 310 as a solution, a suspension, a slurry, or other mixture. In an exemplary embodiment, medication, along with the fluid which carries the medication, diffuses across the hollow fibers of cartridge 320 into the breathing gas, such as oxygen. The breathing gas, now medicated, flows to cannula 332, through which the patient then inhales the medicated breathing gas.

Alternatively, cartridge 320 and/or hollow fiber membranes 322 may be impregnated or coated with medication to be infused with fluid into the breathing gas. The compounds used to manufacture cartridge 320 and the cartridge hollow fibers may be modified to optimize the chemical properties (such as polarity) for impregnating or coating the components with medication. An exemplary embodiment contains polysulphone hollow fibers, although one skilled in the art would also recognize that the fibers may be manufactured from many different materials, including but not limited to, nylon, polypropylene, or cellulosics. As the fluid passes through cartridge 320 and hollow fiber membranes 322, medication from cartridge and/or hollow fiber membranes 322 is entrained in the fluid and infused into the breathing gas.

An alternative device that may be used to deliver medication to a patient through a breathing gas is disclosed in U.S. Pat. No. 4,632,677, issued to Richard H. Blackmer, the disclosure of which is incorporated herein by reference, which describes an oxygen-enriching apparatus including means for increasing or regulating the humidity of the air. The Blackmer apparatus employs an array of membrane cells, a vacuum pump to draw a flow of humidity-and-oxygen-enriched air from each cell, low- and high-temperature condensers connected to receive air drawn from the cells, and a proportioning valve connected to the condensers for providing a desired humidity level of the air.

Another suitable system for the delivery of medication via breathing gas is described in U.S. Pat. No. 4,773,410, issued to Richard H. Blackmer et al., the disclosure of which is incorporated herein by reference. The apparatus described by the Blackmer et al. '410 patent includes a permeable membrane to permit a liquid-vapor boundary, as well as means for delivering a substantially condense-free saturated vapor-gas stream to a respiratory tract. In one embodiment described in the Blackmer et al. '410 patent, the apparatus uses a delivery tube with electrical heating elements that heat the air as it passes through the tube. In another embodiment, a heater heats water which is then delivered through a separate tube that is connected to the delivery tube near the delivery tube's exit port. The heated water then flows counter-current to the air flow to heat the air and exits the delivery tube near its opposite end.

Still another suitable system for the delivery of medication via breathing gas is described in U.S. Patent Publication No. 2003/0209246 A1, the entire disclosure of which is incorporated herein by reference. This publication describes embodiments of an apparatus and method for respiratory tract therapy adapted to heat and humidify air and to deliver heated and humidified air to the respiratory tract of a human patient.

The present invention can be used to treat airflow obstructions and limitations due to mucosal edema, inflammation, bronchoconstriction, and other primary pathophysiologic abnormalities that occur in acute exacerbations of COPD, status asthmaticus, or for delivering pharmaceutical regime throughout management of lung and breathing disorders. According to an exemplary embodiment of a method according to this invention, high flow therapy (HFT) and/or flows delivered at traditional flow rates (2-6 lpm for adults) may be utilized for the inhalation of medications such as short-acting and long-acting beta₂-agonists, e.g., epinephrine; anticholinergics (bronchodilators); methylxanthines; bacteriophages; and sympathomimetics via the respiratory system.

Alternative embodiments of the present invention include administering medication, the combination of ipratropium bromide and albuterol over an extended duration at an appropriate dosage for the individual. This delivery method provides a mechanism to improve delivery and dosing of a wide range of therapeutic compounds by allowing for continuous delivery of a lower dose than was traditionally possible with other delivery mechanisms. Dosing can be accomplished by varying the concentration of the therapeutic molecule in the solution, the flow rate of the breathing gases, the passage rate of the medicated solution across the fiber membrane, or any combination thereof.

Further, the device or system according to embodiments of this invention are adapted to administer and deliver formulary intended to improve breathing and respiration, cardiac function, rate, rhythm, output, efficiency, and function via the respiratory system. It is also optionally used to warm and deliver mixtures of breathing medical gases to improve cardiac and respiratory function. Still yet another embodiment of the present invention is to deliver medication for the treatment of upper respiratory and breathing conditions associated with rhinitis and sinusitis.

Embodiments of the system and method disclosed herein are configured to deliver a wide variety of medications for treatment of indications including, but not limited to infection, sepsis, or invasion of opportunistic agents into humans and mammals, type I and type II respiratory failure, respiratory distress syndrome, chronic lung disease, inflammation and bronchoconstriction, acute exacerbations of COPD, airway pH management, type I and type II diabetes, and other ailments where small molecule delivery into the upper airway or lungs is indicated for treatment. Examples of such medications include antibiotics, bacteriophage, bacteriopsonin, virucides beta-2 antagonist, anticholigerics, steroids, leukotriene antagonists, acidic and basic molecules, insulin, and surfactants, or any combination thereof. Specific examples include, but are not limited to aminophylline BP93/USP23, bromhexine HCl, clenbuterol, cromolyn sodium, dextromethorphan, guaifenesin, heliox, ketotifen, mesna, salbutamol, theophylline, pulmozyme, acetylcysteine, albuterol, beclomethasone dipropionate, epinephrine racemic, ethanol, halothane, isoetharine, isoproterenol, ipratropium bromide, metaproterenol sulfate, hypertonic saline, isotonic saline, hypotonic saline, nitric oxide, sodium bicarbonate, terbutaline, and engineered genes for gene and small molecule therapy. This list is not necessarily all-encompassing of the many medications that may be delivered to a patient by the device, but is instead representative of the families of medications that can be delivered using a system or method according to this invention.

The exemplary medication delivery systems and methods disclosed herein confer several advantages. For example, it has been recognized that when medications are delivered at room temperature or lower, it may become uncomfortable for a patient. Accordingly, because embodiments of a system or method according to this invention facilitate the delivery of medications at temperatures above room temperature, patient comfort is optionally increased in this manner. The exemplary device, with the potential to operate as a high flow therapy system, is capable of delivering a liquid including one or more medications over an extended period of time via a nasal cannula at Body Temperature Pressure Saturated (BTPS) conditions. The device allows one to regulate dosing by adjusting either the concentration of the drug entrained in the breathing gas or the flow rate of medicated gas being delivered to the patient, or both.

It has been recognized that conventional aerosols used for delivering medications to a patient is often in a large particulate size, which in some cases inhibits effective distribution in the lungs of a patient. Accordingly, because embodiments of a system or method according to this invention facilitate the delivery of medications with a smaller aerosol particulate size as compared to conventional aerosol delivery, effective distribution of the medication in the lung is optionally increased in this manner.

Additionally, inhaled systems sometimes provide gas flow delivery at an inspiratory flow that does not match human breathing demands. Accordingly, because embodiments of a system or method according to this invention facilitate gas flow delivery at an inspiratory flow that can match human breathing demands, treatment is optionally improved in this manner as well.

The system and method of the present invention provide the ability to administer medication to the patient at a continuous rate over an extended period of time. The present invention may thus be used with medication specifically formulated for treatments over an extended period of time. Such medication may be at a lower dose concentration than present medications.

### EXAMPLE

In an example according to the present invention, a stock solution of Paeru-03 phage in a concentration of 8×10¹⁰ plaque forming units per milliliter (PFU/ml) was diluted 100-fold in sterile phosphate-buffered saline (PBS) immediately before the experiment in volumes of 0.1 ml phage and 9.9 ml PBS, resulting in 10 ml of phage solution, at a concentration of 8×10⁸ PFU/ml. Five (5) ml of this solution was further diluted in 495 ml of sterile faucet water, for a 100-fold dilution, resulting in a total volume of 500 ml having a concentration of 6×10⁶ PFU/ml.

The solution was used in a Vapotherm® 2000i system, which was operated for 15 minutes, at 60 ml/hour flow speed, with a temperature gradient of 38 degrees Celsius at an operating pressure of 3-4 psig. The Vapotherm® 2000i system used a vapor transfer cartridge having pore openings sized at about 0.1 micron in diameter. About 1 ml of post-vaporizer solution was collected after a 15 minute run.

The phage concentration was measured at the output at 3×10³ PFU/ml, this demonstrates that phage added to the water reservoir do pass through the cartridge infuser, are entrained in the breathing gases, and are delivered at the end of the delivery circuit.

Although the invention is illustrated and described herein with reference to specific embodiments, the invention is not intended to be limited to the details shown. Rather, various modifications may be made in the details within the scope and range of equivalents of the claims and without departing from the invention. Such variations include, for example, delivering medication to infants via a nasal cannula; delivering medication for the treatment of conditions beyond upper respiratory and breathing conditions associated with rhinitis and sinusitis; and delivering substances to penetrate the production of nitric oxide.

What is claimed:

1. A system for delivering medication to a patient comprising:
an infuser including a membrane, the infuser entraining a fluid from a first side of a membrane and a medication into a gas on a second side of the membrane to form a medicated humidified gas; and
a delivery system coupled to the infuser, the delivery system being configured to deliver the medicated humidified gas to the patient.

2. The system of claim 1, wherein the medication is disposed on the first side of the membrane.

3. The system of claim 1, wherein the medication is disposed on the second side of the membrane.

4. The system of claim 1, wherein the medication is mixed with the fluid.

5. The system according to claim 1, wherein the system is configured to deliver the medicated humidified gas using high flow therapy.

6. The system according to claim 1, wherein the delivery system thermally insulates the medicated humidified gas.

7. The system according to claim 1, wherein a concentration of the medication is determined based on a flow rate of the gas through the infuser.

8. A system for delivering medication to a patient comprising:
an infuser having a membrane, the infuser including a medication that produces a medicated humidified gas for delivery; and
a delivery system coupled to the infuser, the delivery system configured to deliver the medicated humidified gas to the patient.

9. The system of claim 8, wherein the system further comprises:
a fluid source supplying a fluid to the infuser; and
a gas source supplying a gas to the infuser;
wherein the infuser infuses the fluid into the gas using the membrane including the medication to produce the medicated humidified gas.

10. The system according to claim 9, wherein the membrane includes a plurality of porous fibers coated with the medication such that the gas passes through an interior of the porous fibers and the fluid passes over an exterior of the porous fibers, through pores in the porous fibers to the interior of the porous fibers, and over the medication, thereby introducing the fluid and medication to the gas to produce the medicated humidified gas.

11. The system according to claim 8, wherein the medication is provided in an amount depending on a flow rate of the gas through the infuser.

12. A system for delivering medication to a patient comprising:
means for producing a medicated humidified gas for delivery to the patient; and
means for delivering the medicated humidified gas to the patient.

13. A method of delivering medication to a patient comprising:
entraining a fluid from a first side of a membrane and a medication into a gas on a second side of the membrane to form a medicated humidified gas; and
delivering the medicated humidified gas to the patient using a delivery system.

14. The method of claim 13, further comprising:
adding the medication to the fluid; and
infusing the fluid including the medication into the gas to form the medicated humidified gas.

15. The method of claim 13, further comprising heating the fluid prior to introducing the fluid into the gas.

16. The method according to claim 13, further comprising determining a flow rate of the gas and determining an amount of the medication based on the flow rate.

17. The method of claim 13, further comprising:
adding the medication to a humidification cartridge including the membrane; and
infusing the fluid into the gas using the humidification cartridge including medication to form the medicated humidified gas.

18. The method of claim 13 wherein delivering the medicated humidified gas to the patient includes delivering the medicated humidified gas between about 33 and about 43 degrees Celsius at greater than about 95% relative humidity.

19. The method of claim 13, wherein the step of delivering the medicated humidified gas to the patient comprises delivering the medicated humidified gas to the patient via a nasal cannula.

20. A method of delivering medication to a patient comprising:
infusing a fluid and a medication into a gas using a humidification cartridge to form a medicated humidified gas; and
delivering the medicated humidified gas to the patient.

21. The method according to claim 20, further comprising determining a concentration of the medicated gas to be delivered to the patient and determining a concentration of the medication in the infused fluid based on the concentration of the medicated gas to be delivered to the patient.

22. The method of claim 20, wherein the method further comprises, prior to the infusing step, adding the medication to the humidification cartridge.

23. The method according to claim 20, wherein the method further comprises removing the humidification cartridge and replacing the humidification cartridge with a replacement cartridge.

24. The method according to claim 23, further comprising selecting the humidification cartridge having a pore size large enough to pass the medication therethrough.

25. The method according to claim 20, further comprising selecting the humidification cartridge based on the medication for delivery to the patient.
